(19) United States
(12) Patent Application Publication
(10) Pub. No.: US 2023/0363956 A1
(43) Pub. Date: Nov. 16, 2023

(54) MOISTURE DETECTION SYSTEM, METHOD FOR USE OF SYSTEM, AND LAMINATE FOR SYSTEM

(71) Applicant: FLEXIBLE TECHNOLOGIES, INC., Abbeville, SC (US)

(72) Inventors: Ronald L. CARLAY, II, Laurens, SC (US); Hunter FARR, Aiken, SC (US)

(21) Appl. No.: 18/142,676

(22) Filed: May 3, 2023

Related U.S. Application Data
(60) Provisional application No. 63/340,969, filed on May 12, 2022.

(51) Int. Cl. A61F 13/42

(52) U.S. Cl. CPC A61F 13/42; A61F 2013/424

(57) ABSTRACT

A moisture detection system and its method of use include a laminate, the laminate further made up of a pair of electrically conductive layers and a water absorbent material. The water absorbent material is positioned between the pair of electrically conductive layers, at least one of the electrically conductive layers can include openings therein to allow water to pass therethrough or be water impervious such that the edges of the laminate allow for passage of water to the water absorbent material. A capacitance measuring device is provided and connected to the electrically conductive films, the capacitance measuring device including a baseline representing a dry state of the water absorbent material and configured to monitor for the presence of moisture in the water absorbent layer by capacitance measurement across the laminate. Also included is an indicator based on a capacitance measurement that represents moisture in the water absorbent material.

This application claims priority under 35 USC 119(e) based on provisional application No. 63/340,969, filed on May 12, 2022, which is herein incorporated in its entirety.

### FIELD OF THE INVENTION

A moisture detection system includes a laminate, a capacitance measuring device, and an indicator, wherein the laminate includes one or more electrically conductive layers and a water absorbent material therebetween to facilitate moisture detection, the water absorbent layer exposed for moisture detection by openings in one or both of the electrically conductive layers or having the water absorbent material being exposed at the laminate edges for water absorption.

### BACKGROUND ART

It is known to use electrical conductivity and capacitance to monitor the presence of moisture. More particularly, common moisture detection devices that rely on the conductivity of water to complete an open circuit are prevalent in a multitude of applications. Typically, the devices have two contacts that are isolated from each other—often times an absorbent material is placed between them. In the presence of water, the current is allowed to flow from one contact to the other, thereby powering some type of alarm or other kind of indicator.

Also prevalent in the prior art are capacitance sensors which can be used to determine the presence of water by measuring a change in dielectric permittivity. Capacitors contain two electrical conductors (typically plates) separated by a dielectric. The capacitance of the capacitor can be measured for given dielectric conditions (air, dry materials, etc.). As the properties of the dielectric change, the resulting change in the capacitance can be measured and used as a basis for a signal from an indicating device or indicator, e.g., an alarm or the like.

These kinds of prior art devices are most often a discreet sensor that is capable of sensing water in a given location. There are examples of water sensors that cover large areas. This is typically done by enlarging both sets of contacts (wires, printed circuits, etched films, etc.) over the area to be monitored. Often, the network of contacts on one side of the sensor will be slightly off-set from those on the other side to prevent false alarms from the contacts being forced together. Regardless of the size or type of sensors employed, the device components, e.g., the electrical contacts or conductors, etc., are designed into the devices and the devices are integrated into the product or space to be monitored. The electrically conductive contacts serve a singular purpose—to function with any water present as part of an electrical circuit for the purpose of providing a warning. If no water is present, the contacts provide no useful function. The same is true for capacitive sensing—the components of the sensor serve the singular function of sensing the change in the capacitance of the dielectric. The sensors add value only in the event of a “failure” but are still lacking in an ability to provide coverage for moisture detection over large areas that are in need of monitoring.

Water sensors for ducts can be probes, rope sensors, float switches, and others. Again, these are discreet sensors that are added to the duct and function separately from the duct components and materials. Detecting the presence of moisture along the length of the duct as well as anywhere around the circumference would require a very large sensor or many smaller sensors to cover the surface area. Most prior art sensors for ductwork require additional labor and installation steps—especially for sensing the entire surface area or even a large percentage thereof. Therefore, the prior art sensors can be difficult and expensive to apply.

The use of electrically conductive materials part of a duct or pipe is also disclosed in WO 2021/163465 to Carlay et al., which is also incorporated herein by reference. In this publication, leaks in pipes are monitored as part of the pipe construction, which uses capacitance for moisture detection. A pair of electrically conductive materials are used, one either the pipe itself or a perforated electrically conductive layer surrounding the pipe. The other electrically conductive material is separated from the electrically conductive pipe or perforated electrically conductive layer by a dielectric. Capacitance change is monitored for leaks in the pipe.

While leaking pipes are a major source of water damage, other sources of water can also cause damage to a given structure and methods and systems that monitor for pipe leaks are not adaptable for monitoring for moisture detection from other sources. For these sources, the prior art teaches a number of different discrete sensors.

In terms of leaks in connection with structures, the methods currently employed for moisture detection vary from being impractical, tedious and/or time consuming, and not always foolproof. Different ways to check for moisture associated with a particular structure, e.g., behind a wall, in a ceiling and the like, include: (1) a visual approach by looking for staining; (2) an olfactory approach where the sense of smell is used to detect odors that may indicate mold, mildew, or the like; (3) a physical approach where holes are drilled through a structural component and moisture sensing probes are inserted into the holes; (4) a thermal approach where infrared thermal scanning may be employed, and (6) discrete sensors, where a number of discrete sensors are placed at appropriate locations where leaks may occur. These kinds of discrete sensors that use wireless communication, e.g., an IoT sensor, are also problematic in that they may have limited range for detecting moisture. While some of these kinds of sensors try to alleviate any limitation in range by including an appendage, e.g., a rope-like sensor that extends the sensing range in a linear fashion, these kinds of sensors are still unable to monitor wide areas where water leakage can occur, e.g., multiple wall cavities, inner wall surfaces of cladding, sheetrock, and the like, broad sections of roof lines, sections of material around windows and doors, and structures whose peripheries needs to be monitored for moisture.

As such, a need exists to provide improved systems and methods for monitoring moisture in structures, particularly in locations that could be susceptible to water damage, and other locations where water leakage may occur. The locations can be in structural areas of a given structure that are difficult to detect. Because of the difficulty in rapid detection in these locations, leaks can cause cumulative and extensive damage if not quickly detected. Most of the prior art systems for moisture detection are not designed to reliably function in hard-to-detect areas due to cost considerations and system configurations. The present invention responds to this need by providing a moisture detection system that allows for comprehensive moisture detection in structures and is simple in its construction and operation.

### SUMMARY OF THE INVENTION

The invention provides an improved moisture detection system that is flexible, low in cost, and easily sensitive to low levels of moisture to provide an effective indication of the presence of moisture in a location where the system resides.

The invention also provides a method of using the invention system in a desired location for moisture detection.

Another aspect of the invention is a laminate construction that is specially designed to be used in the inventive moisture detection system.

Other objects and advantages of the invention will be apparent from the detailed description provided below.

In the system aspect of the invention, a moisture detection system is provided that includes a laminate, a capacitance measuring device, and an indicator. The laminate includes a pair of electrically conductive layers and a water absorbent material. In one embodiment, the water absorbent material is positioned between the pair of electrically conductive layers, whereby at least one of the electrically conductive layers has openings therein to allow water to pass therethrough. The openings can be through one or both of the electrically conductive layers.

In another embodiment, the openings can extend through both layers and through the water absorbent material. In this configuration, moisture can absorb into the edges of the water absorbent material at the through opening so as to allow moisture detection.

In yet another embodiment, the one or both of the electrically conductive layers would not have any openings therein and detection of moisture can be accomplished by the water absorbent material being exposed at one or more edges of the laminate where the electrically conductive layers and water absorption material terminate. This embodiment is particularly useful if the laminate is configured vertically. In this configuration, moisture could enter the laminate through edges of the laminate for detection thereof.

The capacitance measuring device is connected to the pair of electrically conductive films and it includes a baseline representing a dry state of the water absorbent material. The capacitance measuring device is configured to monitor for the presence of moisture in the water absorbent layer by capacitance measurement across the laminate. One advantage of the laminate construction is that it can be configured in any number of ways, e.g., cut, trimmed, bent, twisted, etc., and still provide a baseline capacitance from which a capacitance change can be monitored. The moisture detection system can include a plurality of discrete sections of laminate, the electrically conductive layers of each discrete section of laminate electrically connected together for moisture detection using the capacitance measuring device.

The indicator is based on a capacitance measurement that represents a presence of moisture in the water absorbent material, such measurement requiring some action to be taken.

The water absorbent material can be attached to the pair of electrically conductive films or float therebetween. While the water absorbent material can be any type that would absorb water for moisture detection using capacitance, preferably, the water absorbent material is selected from the group consisting of paper, paperboard products, and non-woven, woven or knitted natural or synthetic materials.

While the electrically conductive layer can be any material that would conduct electricity for the capacitance measurement, preferably, it can be selected from the group consisting of a metallized polyester film, a conductive scrim, a conductive fabric, a conductive mesh, and a conductive wool.

The water absorbent material can be positioned in spaced apart locations between the electrically conductive films or be arranged in a continuous fashion when positioned between the electrically conductive films.

While the openings can be different sizes and shapes providing they are arranged to allow for moisture to pass through the electrically conductive layer for moisture detection, preferably, the openings are spaced apart as discrete throughholes in the at least one electrically conductive layer. The openings can also be part of the electrically conductive layer that is porous to water. From an area perspective, the openings preferably occupy at least 5% of a surface area of the laminate when in use for moisture detection. For most applications where the orientation of the laminate is such that only one of the electrically conductive layers is likely to encounter moisture that would penetrate the layer, only one of the electrically conductive layers has to allow moisture to pass therethrough as a result of its inherent structure, openings, a mesh, or the like. However, other applications may require that moisture can penetrate both electrically conductive layers.

While the water absorbent material can have any thickness sufficient to keep the electrically conductive materials, isolated, a preferred thickness range is from ⅛ inch to ½ inch.

The capacitance measuring means can be battery powered or connected to a power source, e.g., 110 or 24 volts. While the indicator can be any type that provide an indication of moisture presence, preferred indicators include one or more of an audible alert, a visual alert, and a transmitted signal to signify moisture detection. The visual alert can be one or more of a light, an alert on a display, and a text or email message.

In the method aspect of the invention, moisture can be detected in a space by positioning the inventive device and monitoring for the presence of moisture.

While the space can be any one where moisture can be a problem, a preferred space is within a wall and the laminate is positioned on a bottom surface within the wall. The space can also include spaces within a stud wall, whereby the laminate would extend across at least one stud in the stud wall.

When using the inventive system, the laminate can be made in a roll that is wider and/or longer than a final width and/or length of the laminate and cut to size for installation.

The invention also includes just the laminate assembly of the inventive moisture detection system and all of its features. More particularly, the laminate can be sold separately and assembled in the field with the capacitance measuring system and indicator.

### BRIEF DESCRIPTION OF THE DRAWINGS

FIG. 1 is a schematic view of one embodiment of the inventive moisture detection system.

FIG. 2 is a top view of the laminate of the moisture detection system of FIG. 1.

FIG. 3 shows an alternative laminate that uses a different kind of electrically conductive layer to allow moisture therethrough.

FIG. 4 shows a laminate using a differently configured water absorbent material thereof.

FIG. 5 shows one example of a use of the inventive moisture detection system.

FIG. 6 shows a portion of the use of the moisture detection system shown in FIG. 5.

FIG. 7 shows another embodiment of the invention, wherein discrete laminate sections are connected together for moisture detection.

FIG. 8 shows another embodiment of the invention, wherein the laminate is used in a vertical orientation.

### DETAILED DESCRIPTION OF THE INVENTION

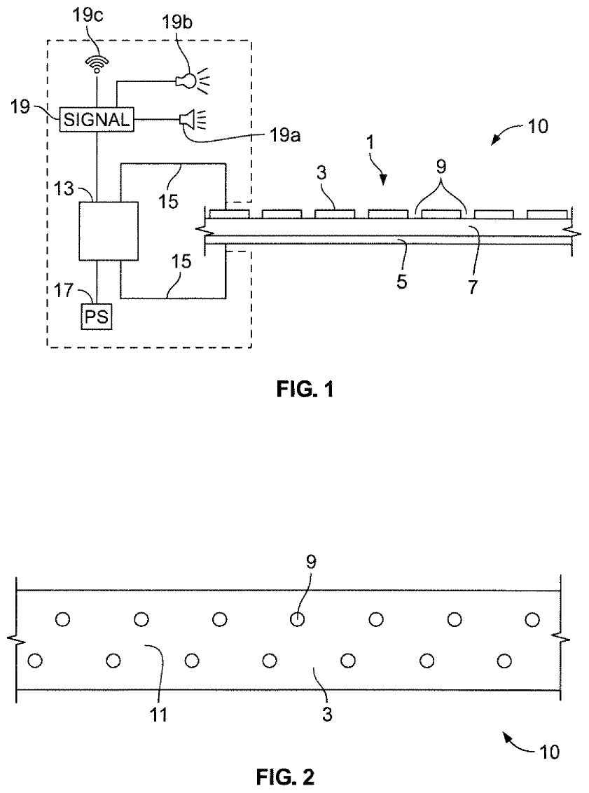

Embodiments of the invention and potential uses are shown in FIGS. 1-8. FIG. 1 shows a schematic view of an exemplary moisture detection system 10 of the invention. FIG. 2 shows a top view of a part of the system of FIG. 1. With reference to FIG. 1, a laminate 1 is provided that includes a pair of electrically conductive material layers 3 and 5, the layers 3 and 5 separated by a water absorbent material 7. In the embodiment of FIG. 1, one of the layers, layer 3 includes a plurality of openings 9. The openings 9 are spaced apart or dispersed across the area of the surface 11 of the layer 3.

The moisture detection system 10 also includes a capacitance measuring device 13, which is electrically connected by conductors 15 to each of the layers 3 and 5 to complete a circuit for capacitance measurement. The capacitance measuring device 13 also includes a power source (PS) designated by reference numeral 17 and, in a general sense, an indicator to alert for moisture detection, the indicator designated by the reference numeral 19. The details of the capacitance measuring device, power source, and indicator are found below.

The openings 9 in the layer 3 allow any moisture/water that may be in the space that the laminate occupies to pass through the electrically conductive material to infiltrate the absorbent material. This accumulation of moisture will then change the capacitance of the laminate and be detected by the capacitance measuring device 13 using a baseline capacitance measurement for the particular laminate construction being used. The baseline capacitance measurement can be stored in the capacitance measuring device or elsewhere and be accessed for use in moisture detection. This baseline measurement is also discussed in more detail below.

Once the capacitance changes, the indicator 19 is triggered so that the appropriate action can be taken, such action dependent on the particular application of the moisture detection system 10.

The indicator 19 can be any type that would provide an indication that the moisture detection system has detected sufficient moisture to warrant an investigation/action. Examples of such indicators are shown in FIG. 1 and they can include those that provide an audible signal 19a, a visual signal 19b, and a transmitted signal 19c to a location remote from the site where the moisture detection is taken place, or combinations thereof. For a transmitted signal, the indicator 19 can include a transmitter to send a signal representing moisture detection to a receiver (not shown). The receiver can be remotely located and configured so that it would receive signals from one or a number of moisture detections in an application wherein a plurality of moisture detection systems are utilized. The indicator 19 could also include text messaging capability or emails, or any other known communication techniques to provide an alert that the system has detected moisture and requires attention. The indicator could be part of the capacitance measuring device itself. For example, if a wireless communication device-using indicator is used, the device could contain the necessary transmitter and other componentry to allow the signal indicating an unacceptable level of moisture is detected to be sent so that the appropriate action could be taken. In other modes, the indicator could be separate, e.g., a separate speaker or visual indicator as a light, and connected to the device for warning purposes. In any configuration, the system needs some indicator capability so that the detection of harmful moisture is known for action-taking purposes.

The electrically conductive layers 3 and 5 that are separated by the absorbent material 7 can be any type that would provide the electrical conductivity for capacitance measurement and can allow the passage of moisture through the material for moisture detection using capacitance measurement. One example of such a layer is a polyester metallized film, which is commonly used in flexible ductwork and the packaging industry. When employing such a film, openings need to be made therein. Other examples of the electrically conductive layers could include a mesh or wool material that is already porous and there is no need to make openings therein. Another embodiment of the electrically conductive layers could be the use of an electrically conductive scrim that would be applied to the absorbent material. Other electrically conductive materials could be conductive fabrics and the like.

The spacing of the openings 9 throughout the surface 11 can vary as can the size of the openings. When using a layer like a polyester metallized film, the openings can be circular in shape and range from 1/16 of an inch to an inch in diameter, with the openings spaced apart by about 1/16 inch to about 2 inches. In another embodiment, the openings can be sized to be at least 5% of the area of a two dimensional electrically conductive material, e.g., a film. In any case, the openings preferences above are based on that which is needed for a laminate in a particular application site to allow for proper moisture detection.

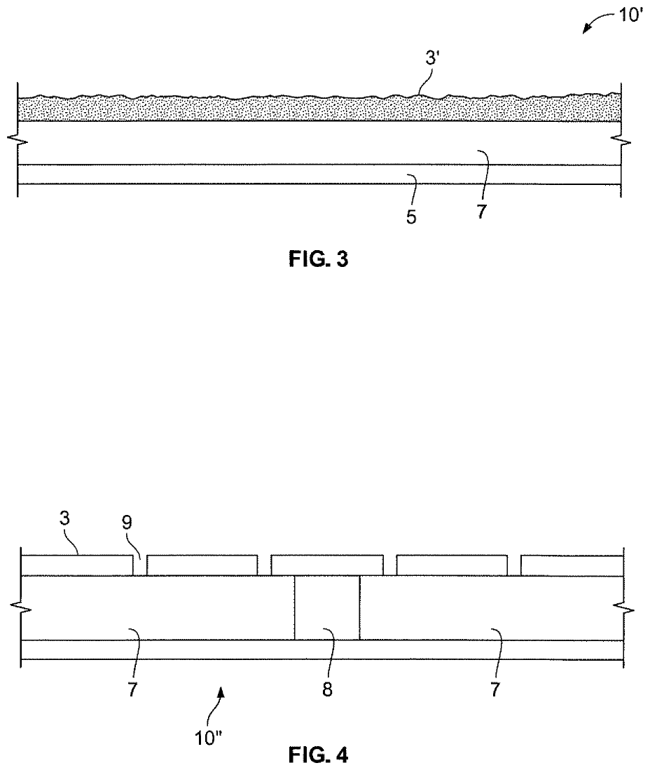

FIG. 3 shows an alternative embodiment of the laminate, which is designated as 10′. In this embodiment, instead of a layer material with discrete openings, a porous metallic material is used and designated by 3′. In the case of using a porous material that may be a mesh or grid like material, e.g., a scrim, or even a three dimensional material like a wool, at least 5% in terms of a porosity is desirable to ensure that, if the presence of moisture is localized, it would still be detected. It is also believed that while forming openings in an electrically conductive material like a film would reduce area and would reduce capacitance, fringe electrical fields around the openings increase fringe capacitance and this is believed to offset any adverse effects by a reduction in the area of the plate, i.e., electrically conductive material. Thus, it is believed that the forming of the openings in the laminate layers does not compromise the moisture detection capability of the system. For most applications where the orientation of the laminate is such that only one of the electrically conductive layers is likely to encounter moisture that would penetrate the layer, only one of the electrically conductive layers has to allow moisture to pass therethrough as a result of its inherent structure, openings, a mesh, or the like. However, other applications may require that moisture can penetrate both electrically conductive layers.

FIG. 4 shows yet another embodiment, designated as 10″. Here, the water absorbent material is not continuous through the length and width of the laminate such that a space 8 exists. The space 8 can span the entire width of the laminate or the space 8 can be distributed throughout the water absorbent material, similar to the distribution for the openings 9 in the electrically conductive material 3 shown in FIG. 2. Having a discontinuous kind of water absorbent material reduces costs as less material is used. However, with the sensitivity of the system, such spaces in the laminate should not affect the ability of the system to detect moisture.

The water absorbent material can be virtually any material that would absorb moisture, including both natural and synthetic water absorbent materials, and allow for moisture detection using capacitance by moisture penetrating the openings in the electrically conductive layer. Examples of such materials are felts, e.g., a non-woven fiber like Modal, which is a type of rayon fabric, polyester, rayon, cotton, wool, etc. Similar material but in woven or knitted form are also candidates for the water absorbent material. Paper and paperboard materials would also work as a water absorbent material.

While the thickness of the absorbent material can vary, it is preferred that the thickness is relatively thin, particularly if the laminate is to be positioned between structures as part of a run thereof as noted below. A thin absorbent material coupled with the kind of metallized polyester film noted above provides a number of benefits. First, cost is reduced as having too much absorbent material is not needed for the capacitance measurement and moisture detection. Second, a relatively thin laminate can be disposed between two structural components, e.g., wall board and a stud of a wall without interfering with the connection between the two components. The absorbent material should be thick enough that if the films are penetrated by a fastener or the like, the films are still spaced apart so that moisture detection can still be accomplished. A preferred minimum thickness for the absorbent material is about ⅛ inch. While there is no theoretical maximum, practically speaking, thicknesses much more than ½ inch don't really add much benefit and can impacts costs and ability to configure the laminate in a desired position or location.

Also, water absorbent layers that are too thick are not as desirable as their drying time is extended. For relatively thin water absorbent layers, these layers can dry out quickly and then be operational again. Also, one can monitor the capacitance measuring device output to allow one to watch the drying out process, i.e., the capacitance measurement drops. If there is no drying out, this is in indication that water is continually present and other remedial action may be necessary.

Moreover, the laminate could have varying thicknesses of the water absorbent material as part of one laminate construction. Depending on a location, there may be areas that are to be covered by one laminate but could have different sensitivities, possible amounts of moisture, and varying drying times following a moisture detection situation. Thus, depending on the particular application, the water absorbent material thickness could be adjusted appropriately. This same variation could apply to the type of water absorbent material. Instead of or with a thickness adjustment, the water absorbent materials that are part of one laminate construction could vary.

In other embodiments, the water absorbent material could be positioned between the electrically conductive materials in a spaced apart manner, e.g., 3-6 inches. In this way, the cost of the laminate is reduced by using less material while not jeopardizing the moisture detection capability of the system.

The water absorbent material and electrically conductive layers can be attached together using any known means, adhesives, bonding techniques, mechanical means, or combinations thereof. In some instances, the water absorbent material could be positioned between the electrically conductive layers and attached together during manufacture of the laminate. In other instances, the water absorbent material could be positioned between the two layers in a temporary fashion, for example, adhesive tape could be used along side edges of the laminate. More particularly, the tape could adhere to the outer surface of each of the two layers, either continuously along the side edge or at spaced intervals along the side edges. In this way, the positioned water absorbent material would be held in place as the tape attachment would prevent the water absorbent material from sliding out from in between the two electrically conductive layers. The tape could remain as a form of securing the laminate together or be removed at an application site, wherein the laminate as a whole be secured in place to keep the water absorbent layer positioned between the two electrically conductive layers.

The inventive moisture detection system can have any number of applications. One application would be to place the laminate of the system at the bottom of a wall cavity, where water is likely to be encountered. The laminate could be just placed at the bottom of the wall cavity or fixed in place. The water absorbent material positioned between the two electrically conductive layers of the laminate maintains an electrical isolation or separation between the layers so that the laminate could also be fastened in place using any kind of mechanical means, stapling, nails, and the like. The laminate could also be glued in place as well. In fact, any space that would need moisture detection can receive the laminate of the invention. By virtue of the construction of the laminate and the easy ability to make the laminate in large width and length dimensions, it can be sized to fit virtually any space, e.g., a narrow slot-like space, or an expansive space. The laminate could be put under sinks, hot water tanks, washing machines, pipes, dishwasher connections, refrigerator connections, washing machine connections, stacked wet walls, or other components that may leak water that could cause damage to a given structure.

In certain applications, the potential for moisture could come below or the side rather than above as would be the case for the application in FIGS. 4 and 5. In these applications, the laminate could be oriented vertically, or horizontally with the perforation facing downward rather than the upward orientation that is shown in FIG. 5. In any event, the laminate needs the surface 11 to be exposed so that the openings 9 can allow moisture to penetrate the openings and be absorbed by the water absorbent layer. As the laminate is not designed for any insulating purposes, any insulating layers on top of the electrically conductive layers is unnecessary and actually prevents the system from properly functioning as it may interfere with moisture absorption by the water absorbent layer 7.

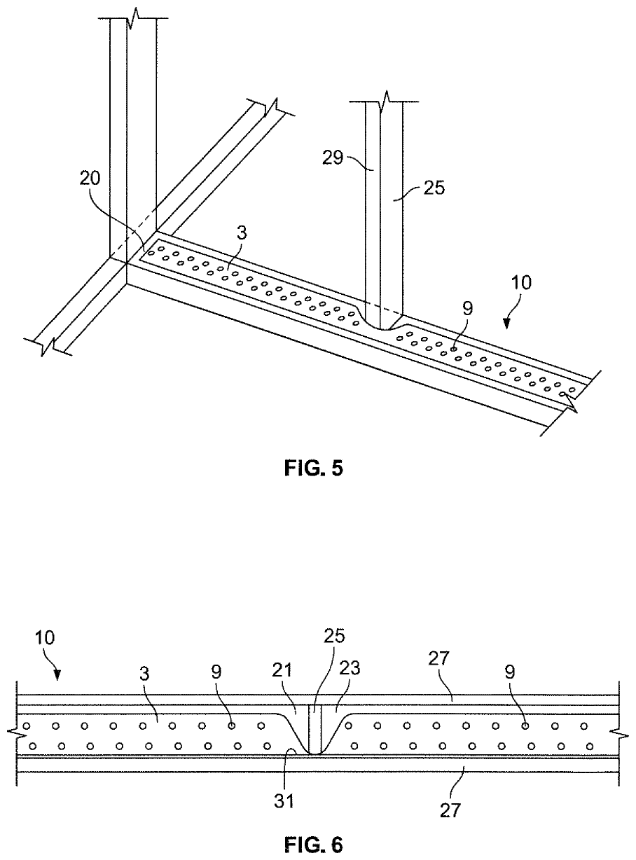

Given the relatively thin dimension of the laminate, one section can be used in multiple wall cavities. This application is shown in FIGS. 5 and 6, wherein the laminate 10 is shown placed on a sill board 20 that is at the bottom of two wall cavities 21 and 23 (FIG. 5), that are separated by a stud 25 and formed in part by two walls 27. The laminate could be attached to the sill 20 using a known attachment means or just laid flat thereon. For illustration purposes, the walls 27 enclosing the cavities 21 and 23 are not shown in FIG. 5 but shown in FIG. 6. When extending between the two wall cavities 21 and 23, the laminate, by virtue of its small thickness, can be turned so as to be in a vertical orientation and run along a vertical side surface 29 of the stud 25 and be positioned between the side surface 29 and an inner surface 31 of the wall 27. Starting with the laminate in space 21, once it passes by the stud 25 in the vertical orientation, the laminate 10 is turned again from its vertical orientation when adjacent the stud side surface 29 so that the laminate 10 lays flat on the bottom of the second wall cavity 23. While only two wall cavities are shown for monitoring purposes, many more than two can be monitored by providing more length of laminate so as to permit extension of the laminate across more studs. An alternate use of the laminate would be to keep the laminate vertically aligned (on its side) and then attach it to a vertical surface for moisture detection. An example of this would be to attach the laminate to an inside surface of a wall or cladding forming a first wall cavity, wrap the laminate around the three sides of a stud positioned between the first wall cavity and an adjacent wall cavity and then attach the laminate to the wall forming the adjacent wall cavity. In this use, there would be no need for a twisting of the laminate as is the case if the laminate is used in a horizontal orientation.

The capacitance measuring device is not shown in FIGS. 5 and 6 but it could be attached to either of the laminates illustrated therein. In applications wherein the laminate structure may be compressed, i.e., between a stud and wall, the baseline capacitance measurement is established after such an installation. In this way, the baseline capacitance and performance of the system for the compressed laminate that is positioned in spaces for moisture detection reflects the compression of the laminate.

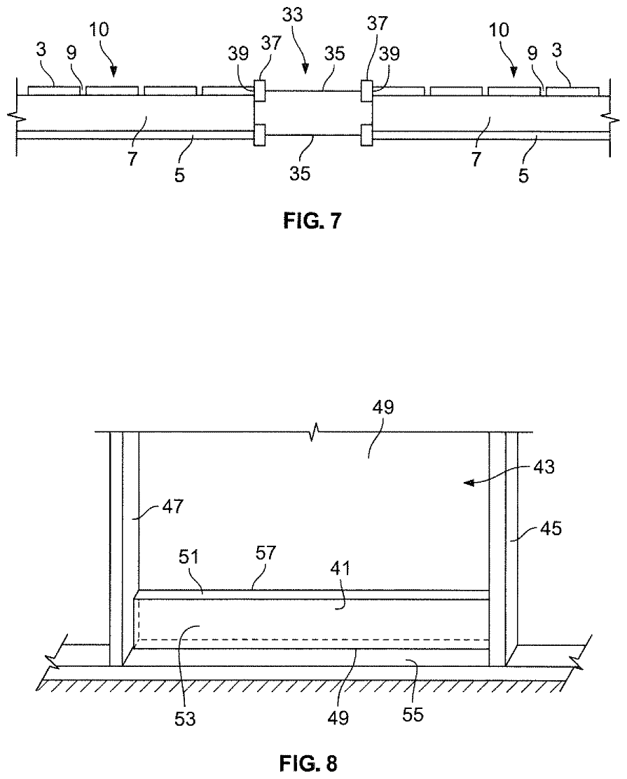

In yet another application, discrete lengths of the laminate can be electrically coupled together to monitor a plurality of discrete spaces. The electrical coupling of the electrically conductive layers can be any known coupling. FIG. 7 shows on example of a pair of laminate sections 10 of a set length and an electrical connection designated by the reference numeral 33 therebetween. In this example, each electrical connection includes an insulated wire 35 and an attachment to the wire, which is generically designated by the reference numeral 37, to a portion 39 of each of the electrically conductive layers 3 and 5 of each laminate section 10. The attachment is shown as a box to represent any known kind of electrical connection. Examples of a removable connection could be an alligator clip that clips to each portion 39 of the electrically conductive layer. The alligator clip could have teeth or just use a flat surface to connect to the electrically conductive layer. Other connections could include a more permanent connection like a soldered connection or a more secure but still removable male-female clip connection as are well known in the art. In fact, any known connection, whether permanent or attachable and detachable, can be employed when coupling adjacent laminate sections together for moisture detection. Having combinable but discrete sections of the laminate, more complex spaces and those with varying shapes could be more easily monitored. For example, two separate spaces in need of moisture detection can be monitored using two discrete sections, that are coupled together by the electrical conductors connecting the two discrete sections together.

Another embodiment of the invention is shown in FIG. 8. Here, the inventive laminate, designated by reference numeral 41, sits in a wall cavity 43 formed by two studs 45 and 47, a back wall 49, and front wall (not shown for clarity purposes). The laminate 41 is positioned with one of its side or lateral edges 49, so that the electrically conductive layers, one shown as 51 is vertically oriented such that a face 53 thereof is perpendicular to the sill member 55.

In this embodiment, the openings described above in the electrically conductive layers are not employed. One reason for this is that the laminate 41 effectively still has an opening in that the water absorbent material between the electrically conductive layers is exposed along the upper side edge 57 of the laminate 41. With this exposure, water running down the wall 49 could flow in between the two electrically conductive layers and be absorbed by the exposed water absorbent material at the side edge of the laminate and be detected using the capacitance measuring device.

In yet a further embodiment, one section of laminate could be configured with openings as disclosed above along a portion of the laminate with the remaining section of laminate not having any openings in the electrically conductive layers. With this configuration, the same laminate could be located in one area where one section of the laminate is essentially horizontal and then the laminate would be located in another area where the laminate could be vertically oriented as shown in FIG. 8 and not be in need of openings in the electrically conductive layers. Further yet, the laminate could be constructed with alternating sections, one with openings in the electrically conductive layer, another adjacent section without the openings, and yet another adjacent section with openings. This is just another example of the flexibility of the laminate construction to be adapted for different locations for moisture detection.

While the laminate shown in FIG. 8 is a rectangular one such that the longitudinal edge is the primary opening for water absorption, the edge best located for water absorption could be different from the longitudinal edge shown in FIG. 8 based on the location where the laminate is placed for moisture detection. Water ingress could be possible from two different directions such that the short or lateral edge of the laminate is aligned horizontally and the long edge would be aligned vertically.

Another aspect of the moisture detection system is the means or means in which capacitance can be measured and monitored once the system is put in a desired location for moisture detection. Virtually any kind of device or means that, once connected to each of the electrically conductive materials of a laminate, has the ability to measure capacitance can be used in the inventive system. The capacitance measuring device will also include a baseline capacitance measurement of the laminate in a dry state. This baseline measurement will then be used once the moisture detection system is in its desired location to determine changes in capacitance and a resulting detection of moisture. The baseline capacitance measurement can be done as part of manufacturing the laminate and prior to its use in the field or be done in the field and before the system is put into operation. Preferably, the baseline capacitance measurement is done in the field as this would more accurately represent the environment of the field site.

In terms of the capacitance sensing circuit, a dry situation is generally reflected by measurements that are below 0.01 micro-farads. However, it is believed that when small quantities of water would enter the space between the two electrically conductive materials, e.g., a teaspoon, the capacitance measurement would jump to 5.0 or more micro-farads. Thus, the capacitance sensing circuit is fully capable of determining a leak in an inner conduit, including the presence of small leaks. Thus, any device able to measure capacitances above 0.01 micro-farads is believed to be satisfactory for the capacitance measuring device.

The capacitance measuring device can be provided with any type of power source for its operation. Depending on the location of the system, the power source could be batteries if the device is easily accessible so that the batteries can be changed when necessary. Also, a 110 volt or a low voltage, e.g., 24-volt, power supply could be used to power the device. The low voltage power supply makes installation at a field site easier in terms of avoiding the necessary code requirements when running 110-volt wiring. The power source 17, if batteries, can be part of the capacitance measuring device 13, or separate therefrom. Similarly, the indicator 19 and its capabilities could be an integral part of the capacitance measuring device or separate therefrom.

In terms of a connection between the capacitance measuring device and the laminate, any known kind of connection could be used, similar to that described above for the connection between laminate sections.

The laminate of the moisture detection system can be made to size for a particular application or made in a bulk size with larger widths and lengths than needed in a field application and cut to size for a particular application. The simplicity of the laminate construction provides a significant advantage in that a large quantity of the laminate as a roll can be made at one time. If a film is used, the roll making can include a perforating step as a part thereof. The opening-containing laminate roll can then be slit to width and/or cut to length for a desired application. Any known process for making the laminate can be employed to make the moisture detection system. When making the laminate in bulk to have a width and length and be later cut for a particular application, a preferred minimum width would be about 0.5 inches. Making the laminate in more narrow widths is not preferred as it tends to limit the ability to customize the bulk laminate for a given application. The laminate could be made in any length, including long lengths, i.e., as many feet of laminate could be stored on a roll for later dispensing and use. Other shapes for the laminate would include rings, squares, circles. Having a laminate in a circular kind of form allows placement of the laminate to surround a roof, wall, or floor penetration as these locations can be susceptible to water ingress.

A significant advantage when producing the laminate in widths and lengths is that a wide area of coverage for moisture detection can be obtained as opposed to laminates as more discrete elements, e.g., discs, as a large number of discs would be required to cover an area of concern. Likewise, the prior art ropes require significant lengths of rope for coverage and this need is alleviated by the use of the large surface area laminate.

From a width standpoint, the laminate could have any desired width that would accommodate the space desired for moisture detection. In a wall cavity, the laminate width could match the distance between walls, e.g., 3.5 inches, when using a 2×4 sill member, or 5.5 inches when using a 2×6 sill member. Again, the laminate could be made in these widths or made wider and cut to size for a given application.

In one exemplary use, the laminate could be manufactured first in a stock size and shipped to an application site for further cutting to size or manufactured in a custom size designed to be used as is at the application site.

The laminate could be sent to the application site with the capacitance measuring device and the indicator as a complete system, where the system just needs to be put in place for moisture detection. Alternatively, the laminate and capacitance measuring device and indicator could be connected at the application site. In this mode, the laminate is made on its own for use with a capacitance measuring device and indicator, which could be supplied by the laminate maker or provided by another supplier independent of the laminate maker.

As such, an invention has been disclosed in terms of preferred embodiments thereof which fulfills each and every one of the objects of the present invention as set forth above and provides a new and improved moisture detection system and method of use.

Of course, various changes, modifications and alterations from the teachings of the present invention may be contemplated by those skilled in the art without departing from the intended spirit and scope thereof. It is intended that the present invention only be limited by the terms of the appended claims.

I claim:

1. A moisture detection system comprising
a) a laminate, the laminate further comprising a pair of electrically conductive layers and a water absorbent material, the water absorbent material positioned between the pair of electrically conductive layers, and optionally, at least one of the electrically conductive layers including openings therein to allow water to pass therethrough,
b) a capacitance measuring device connected to the pair of electrically conductive layers, the capacitance measuring device including a baseline representing a dry state of the water absorbent material and configured to monitor for the presence of moisture in the water absorbent layer by capacitance measurement across the laminate; and
c) an indicator based on a capacitance measurement that represents a presence of moisture in the water absorbent material.

2. The moisture detection system of claim 1, wherein the water absorbent material is attached to the pair of electrically conductive layers.

3. The moisture detection system of claim 1, wherein the water absorbent material is selected from the group consisting of paper, paperboard products, and non-woven, woven or knitted natural or synthetic materials.

4. The moisture detection system of claim 1, wherein each electrically conductive layer is selected from the group consisting of a metallized polyester film, a conductive scrim, a conductive fabric, a conductive mesh, and a conductive wool.

5. The moisture detection system of claim 1, wherein the water absorbent material is positioned in spaced apart locations between the electrically conductive films or is continuous when positioned between the electrically conductive films.

6. The moisture detection system of claim 1, wherein the openings are spaced apart as discrete throughholes in the at least one electrically conductive layer.

7. The moisture detection system of claim 1, wherein at least one of the electrically conductive layers include openings therein to allow water to pass therethrough.

8. The moisture detection system of claim 7, wherein the openings are in the at least one electrically conductive layer that is porous to water.

9. The moisture detection system of claim 7, wherein the openings occupy at least 5% of a surface area of the laminate when in use for moisture detection.

10. The moisture detection system of claim 1, wherein the water absorbent material ranges in thickness from ⅛ inch to ½ inch.

11. The moisture detection system of claim 1, wherein the capacitance measuring means is battery powered or connected to a power source.

12. The moisture detection system of claim 1, wherein the indicator provides one or more of an audible alert, a visual alert, and a transmitted signal to signify moisture detection.

13. The moisture detection system of claim 1, wherein visual alert is one or more of a light, an alert on a display, and a text or email message.

14. The moisture detection system of claim 1, comprising a plurality of discrete sections of laminate, the electrically conductive layers of each discrete section of laminate electrically connected together for moisture detection using the capacitance measuring device.

15. A method of detecting moisture in a space comprising:
a) providing the laminate of claim 1; and
b) positioning the laminate in the space and monitoring for the presence of moisture.

16. The method of claim 15, wherein the space is within a wall and the laminate is positioned on a bottom surface within the wall.

17. The method of claim 15, wherein the space includes spaces within a stud wall, and the laminate extends across at least one stud in the stud wall.

18. The method of claim 15, wherein the laminate is obtained from a roll of laminate that is wider and/or longer than a final width and/or length of the laminate and cut to size for installation.

19. A laminate assembly for a moisture detection system comprising a laminate, the laminate further comprising a pair of electrically conductive layers and a water absorbent material, the water absorbent material positioned between the pair of electrically conductive layers, optionally, at least one of the electrically conductive layers including openings therein to allow water to pass therethrough, the electrically conductive layers adapted to be connected to a capacitance measuring device to permit moisture detection by moisture penetrating the openings in the at least one conductive layer.

19. The laminate of claim 18, wherein the water absorbent material is selected from the group consisting of paper, paperboard products, non-woven, woven or knitted natural or synthetic materials.

20. The laminate of claim 18, wherein the water absorbent material is attached to the pair of electrically conductive films.

21. The laminate of claim 18, wherein water absorbent material is positioned in spaced apart locations between the electrically conductive films or is continuous when positioned between the electrically conductive films.

22. The moisture detection system of claim 1, wherein at least one of the electrically conductive layers include openings therein to allow water to pass therethrough.

23. The laminate of claim 22, wherein the openings are spaced apart as discrete throughholes in the at least one electrically conductive layer.

24. The laminate of claim 22, wherein the openings are in the at least one electrically conductive layer that is porous to water.

25. The laminate of claim 22, wherein the openings occupy at least 5% of a surface area of the laminate when in use for moisture detection.

26. The laminate of claim 19, wherein the water absorbent material ranges in thickness from ⅛ inch to ½ inch.

27. The laminate of claim 19, wherein the laminate is made with a width and length and the width is at least 0.5 inches.
